(19) United States
(12) Patent Application Publication
(10) Pub. No.: US 2020/0057083 A1
(43) Pub. Date: Feb. 20, 2020

(54) IMMUNOASSAY SYSTEM CAPABLE OF SUGGESTING ASSAYS BASED ON INPUT DATA

(71) Applicant: HYCOR Biomedical, LLC, Indianapolis, IN (US)

(72) Inventor: Mark David Van Cleve, Garden Grove, CA (US)

(21) Appl. No.: 16/346,741

(22) PCT Filed: Oct. 27, 2017
(86) PCT No.: PCT/US2017/058845

§ 371 Date: May 1, 2019

Related U.S. Application Data
(60) Provisional application No. 62/415,876, filed on Nov. 1, 2016.

(51) Int. Cl. G01N 35/00; G01N 33/543; G16C 20/10; G16H 50/20; G06F 9/451; G16H 70/60; G16H 10/40

(52) U.S. Cl. CPC G01N 35/0098; G01N 33/54333; G01N 33/54366; G16H 10/40; G16H 50/20; G06F 9/451; G16H 70/60; G16C 20/10

(57) ABSTRACT

Methods and apparatus for performing diagnostic assays using recommendations based on one or more user input are described herein. In an embodiment, a system for diagnosing and testing a patient for one or more disease includes an assay apparatus configured to perform a plurality of different assays on a patient sample using a plurality of stored capture reagents, a user interface in operable communication with the assay apparatus, the user interface configured to allow input of a patient's symptoms, and a control unit in operable communication with the user interface, the control unit configured to (i) analyze a disease profile database based on the symptoms inputted into the user interface, (ii) output at least one recommended assay based on the analysis of the disease profile database, and (iii) cause the assay apparatus to perform the at least one assay using at least one of the plurality of capture reagents.

### CROSS-REFERENCE TO RELATED APPLICATIONS

The present disclosure is related to PCT/US2016/043873, filed Jul. 25, 2016, entitled, “On-Board Kitting,” the entire disclosure of which is hereby incorporated by reference herein.

### FIELD OF THE DISCLOSURE

The present disclosure relates generally to methods and apparatuses for suggesting diagnostic assays based on input data, and more specifically to an apparatus capable of diagnosing potential diseases based on a patient's symptoms or other data, suggesting assays to test for the potential diseases, and mixing a plurality of individual capture reagents to run the suggested assays.

### BACKGROUND OF THE DISCLOSURE

Many immunochemistry analysis systems run assays by analyzing whether analyte molecules in a patient's biological sample (e.g. serum or plasma) attach to paramagnetic particles. To bind analyte molecules of interest to the paramagnetic particles, a capture reagent is first bound to the paramagnetic particles, and then the patient sample is bound to the capture reagent.

The capture reagents of such systems, however, are used individually as opposed to being combined, and the selection of each individual capture reagent is determined by the individual running the assay. For the individual to appropriately evaluate what capture reagent should be used to test a patient sample on-the-fly for a particular disease symptom, the individual must have in-depth knowledge of thousands of potential symptoms and corresponding diseases. The individual must also have knowledge of symptoms that can be the result of multiple diseases and know which capture reagents to test for each of the possible diseases. The individual running the assay, however, may not be a doctor, and even if the individual is a doctor, it would be very difficult for the doctor to make on-the-fly decisions regarding which capture reagents to use to run assays for all possible diseases corresponding to particular symptoms.

### SUMMARY OF THE DISCLOSURE

Described herein are methods and apparatus for performing diagnostic assays using recommendations based on one or more user input. In a general example embodiment, a system for diagnosing and testing a patient for one or more disease includes an assay apparatus storing a plurality of capture reagents, the assay apparatus configured to perform a plurality of different assays on a patient sample using the plurality of capture reagents, a user interface in operable communication with the assay apparatus, the user interface configured to allow input of at least one patient symptom, and a control unit in operable communication with the user interface, the control unit configured to (i) analyze a disease profile database based on the at least one patient symptom inputted into the user interface, (ii) output at least one recommended assay using at least one of the plurality of capture reagents stored by the assay apparatus based on the analysis of the disease profile database, and (iii) cause the assay apparatus to perform the at least one assay using the at least one of the plurality of capture reagents.

In another example embodiment, the control unit is configured to cause the assay apparatus to perform the at least one assay by: (i) isolating the at least one of the plurality of capture reagents based on the at least one recommended assay; (ii) binding the at least one of the plurality of capture reagents to paramagnetic particles, (iii) binding analyte molecules from the patient sample to the at least one of the plurality of capture reagents, and (iv) analyzing the bound analyte molecules from the patient sample to determine a positive or negative result for the at least one recommended assay.

In another example embodiment, the at least one disease profile database is configured to output a plurality of recommended assays to be performed by the assay apparatus using two or more of the plurality of capture reagents stored by the assay apparatus.

In another example embodiment, the controller is configured to cause the assay apparatus to perform each of the plurality of recommended assays separately.

In another example embodiment, the controller is configured to cause the assay apparatus to perform each of the plurality of recommended assays simultaneously.

In another example embodiment, the controller is configured to cause the automated immunochemistry analyzer to: (i) mix the two or more of the plurality of capture reagents together; (ii) bind the mixture of the two or more of the plurality of capture reagents to the paramagnetic particles, (iii) bind analyte molecules from the patient sample to the bound mixture of the two or more of the plurality of capture reagents, and (iv) analyze the bound analyte molecules from the patient sample.

In another example embodiment, each capture reagent is specific for an immunogen selected from the group consisting of allergens, infectious disease antigens and autoantigens.

In another example embodiment, the control unit stores locations of the plurality of capture reagents within the assay apparatus, and controls the assay apparatus to perform the at least one assay by retrieving the at least one of the plurality of capture reagents from a corresponding stored location.

In another general example embodiment, a method for diagnosing and testing a patient for one or more disease includes inputting a patient's symptoms into a user interface, analyzing at least one patient symptom by accessing a disease profile database, displaying on the user interface at least one disease that may be present in the patient based on the analysis of the at least one patient symptom, requesting at least one test be performed on a patient sample to test for the at least one disease, receiving a result of the at least one test, and treating the patient for the at least one disease if the result of the at least one test indicates that the patient sample tested positive for the at least one disease.

In another example embodiment, inputting the patient's symptoms into the user interface includes answering questions about the patient prompted on the user interface.

In another example embodiment, displaying on the user interface at least one disease includes displaying on the user interface a plurality of diseases, and performing the at least one assay includes mixing a plurality of capture reagents and performing a plurality of assays simultaneously to determine the occurrence of the plurality of diseases.

In another general example embodiment, a system for diagnosing and testing a patient for one or more disease includes a user interface configured to allow input of at least one patient symptom, a disease profile database stored on a non-transitory computer readable medium, the disease profile database linking a plurality of diseases to a plurality of patient symptoms, and a control unit configured to (i) analyze the disease profile database using the inputted at least one patient symptom, (ii) cause the user interface to display at least one recommended disease to test for the patient based on the analysis, and (iii) allow selection of the at least one recommended disease via the user interface for further testing.

In another example embodiment, the control unit is configured to determine at least one capture reagent to be mixed with a patient sample to perform at least one assay to test for the at least one disease.

In another example embodiment, the control unit is configured to cause the user interface to display a plurality of recommended diseases to test for the patient based on the analysis.

In another example embodiment, the control unit is configured to cause an assay apparatus to test for the plurality of recommended diseases simultaneously by mixing a plurality of capture reagents together.

In another example embodiment, the user interface is configured to allow selection of one or more tests for one or more of the plurality of recommended diseases.

In another example embodiment, the user interface is configured to indicate that one of the plurality of recommended diseases is more likely to be present than another of the plurality of recommended diseases based on the inputted at least one patient symptom.

In another example embodiment, the control unit determines the likelihood of the plurality of recommended diseases based on weights assigned to patient symptoms within the disease profile database.

In another example embodiment, the system includes an automated immunochemistry analyzer, and the control unit is configured to control the automated immunochemistry analyzer to perform the further testing for the at least one disease using at least one capture reagent stored by the immunochemistry analyzer.

In another example embodiment, the control unit stores locations of a plurality of capture reagents within the automated immunochemistry analyzer, and controls the automated immunochemistry analyzer to test for the at least one disease by retrieving the at least one capture reagent from a corresponding stored location.

In another general example embodiment, a method for diagnosing and testing a patient for one or more disease includes inputting a patient's symptoms into a user interface, analyzing at least one patient symptom by accessing a disease profile database, displaying on the user interface at least one disease that may be present in the patient based on the analysis of the at least one patient symptom, determining at least one assay to be performed on a patient sample to test for the at least one disease, selecting at least one capture reagent from a plurality of selectable capture reagents to test for the at least one disease, and performing the at least one assay using the at least one capture reagent.

In another example embodiment, performing the at least one suggested assay includes adding the at least one capture reagent to a container containing paramagnetic particles, binding the at least one capture reagent to the paramagnetic particles, binding analyte molecules from the patient sample to the at least one capture reagent, and analyzing the bound analyte molecules from the patient sample to determine a positive or negative result for the at least one assay.

### BRIEF DESCRIPTION OF THE DRAWINGS

Embodiments of the present disclosure will now be explained in further detail by way of example only with reference to the accompanying figures, in which:

FIG. 1 is a top plan view of an example embodiment of an automated immunochemistry analyzer and reagent system according to the present disclosure;

FIG. 2 is a schematic view of an example embodiment of a control unit associated with the automated immunochemistry analyzer and reagent system of FIG. 1;

FIG. 3A illustrates an example embodiment of a disease profile database that can be stored on and/or accessed by the control unit of FIG. 2;

FIG. 3B illustrates another example embodiment of a disease profile database that can be stored on and/or accessed by the control unit of FIG. 2;

FIG. 4 illustrates another example embodiment of a disease profile database that can be stored on and/or accessed by the control unit of FIG. 2;

FIG. 5 illustrates another example embodiment of a disease profile database that can be stored on and/or accessed by the control unit of FIG. 2;

FIG. 6 illustrates another example embodiment of a disease profile database that can be stored on and/or accessed by the control unit of FIG. 2; and

FIG. 7 illustrates an example embodiment of a control method that can be performed by the control unit of FIG. 2.

### DETAILED DESCRIPTION

Before describing in detail the illustrative system and method of the present disclosure, it should be understood and appreciated herein that the present disclosure relates to methods and apparatuses that prepare, in situ, analytical substrates by mixing a plurality of individual capture reagents to form analytical substrates to optimize diagnostic assays for different types of analyte molecules of interest, specifically for molecules that bind to immunogens. In general, the system utilizes common paramagnetic particles, for example magnetic beads or microparticles, that are pulled to the wall of a reaction cuvette by magnets during a washing process so that liquid can be aspirated from the cuvette. As used herein, the term “in situ” refers to the preparation of the analytical substrate by the system and apparatus disclosed herein and specifically excludes the preparation of analytical substrates manually. As used herein, the term “immunogen” refers to an antigen to which an individual will make a detectable immune response. For the purposes of the present disclosure, immunogen-binding molecules present in the blood of patients are tested for binding to the immunogen. Exemplary immunogens include, but are not limited to, allergens, infectious disease antigens, and autoantigens. Immunogens can be proteins, glycoproteins, carbohydrates, lipids, glycolipids, or nucleic acids. Additionally, the term “immunogen” can refer to a fragment of one of the autoantigens, allergens, or infections agent antigens disclosed herein. As used herein, the term “analytical substrate” refers to a complex of one or more capture reagents and paramagnetic particles. The analytical substrate is substantially free of unbound capture reagent.

As explained in more detail below, using the illustrative system and method of the present disclosure, paramagnetic particles can be coated with one or more capture reagent that will eventually bind analyte molecules of interest in the patient's blood sample. In exemplary embodiments, the capture molecule is an immunogen which binds an immunogen-binding molecule (analyte), such as an antibody, in the patients' blood sample. After the capture reagents bind to the paramagnetic particles and the cuvettes undergo a washing process, the patient sample, and optionally a diluent if needed, is added to the particles in the reaction cuvette and incubated. This allows analytes of interest in the patient's blood sample to bind to the one or more capture reagent that have in turn been bound to the surface of a paramagnetic particle. After the patient sample incubation period, another washing process is performed to remove any excess or unbound sample, and then a conjugate and a luminescent label are added to the cuvette. When added to the cuvette, it can be expected that some portion of the conjugate will bind to the capture reagent/sample complex on the paramagnetic particles after an incubation period. The particles then undergo another wash process to remove any unbound conjugate, and then a luminescent label is added to the reaction cuvette and incubated for a short period of time to allow the chemiluminescent reaction to reach equilibrium. After equilibrium is reached, luminescence and fluorescence readings of the sample can be taken to determine as positive or negative result for the assay.

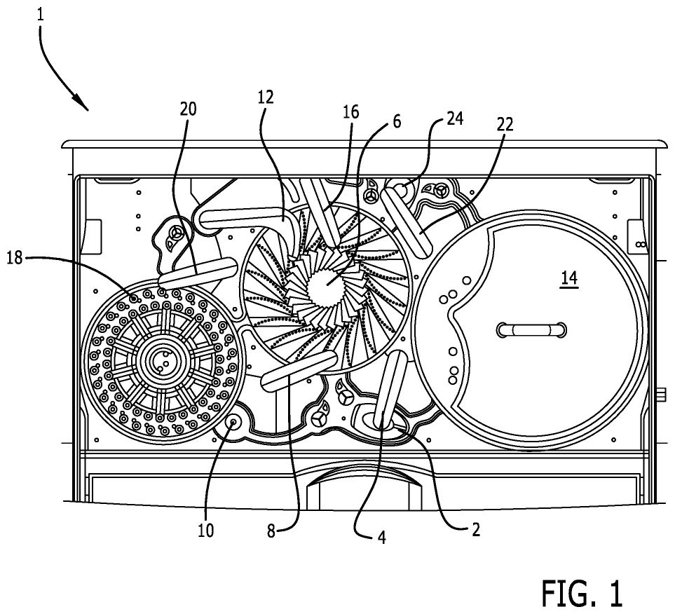

FIG. 1 illustrates various components of an embodiment of an automated immunochemistry analyzer 1 according to the present disclosure. Automated immunochemistry analyzer 1 can take an analyte sample, create an environment that will allow it to bind to a paramagnetic particle, perform a number of washing steps, and then quantify and normalize the luminescence signal of the analyte sample. This can be accomplished through an automated process that utilizes a vortexer 2, an R1 pipettor 4, a reaction rotor 6, an optics pipettor 8, an optics device 10, a multi rinse pipettor 12, a reagent rotor 14, a single rinse pipettor 16, a sample rotor 18, a sample pipettor 20, an R2 pipettor 22, and a mixed substrate container 24.

In one embodiment disclosed herein, an apparatus such as automated immunochemistry analyzer 1 can quantify and normalize the luminescence signal of an analyte sample before reaction of the analyte with the capture reagent. In an embodiment, automated immunochemistry analyzer 1 begins by first dispensing fluorescently labelled paramagnetic particles, or fluo-beads, into a cuvette located within the reaction rotor 6. The fluo-beads can be initially located in vortexer 2 and transferred to reaction rotor 6 by R1 pipettor 4. R1 pipettor 4 can aspirate a desired quantity of the fluo-bead mixture and transfer the aspirated quantity to reaction rotor 6 where it is injected into the cuvette of reaction rotor 6. Optics pipettor 8 can then aspirate a test sample from the cuvette of reaction rotor 6 and transfer the test sample to optics device 10, where fluorescence and luminescence measurements can be recorded. The initial recording of the fluorescence and luminescence signal can be used as a baseline measurement for the initial concentration of fluo-beads in a sample. After recording the measurements, multi rinse pipettor 12 can rinse the cuvettes using a wash buffer.

In order to prepare the analytical substrates, fluo-beads can be transferred from vortexer 2 to a cuvette in reaction rotor 6 via R1 pipettor 4. Reagent rotor 14 contains a plurality of different capture reagents that can be used to run different assays related to different diseases. R1 pipettor 4 can also aspirate one or more capture reagent from reagent rotor 14 and inject the one or more capture reagents into the cuvette located in reaction rotor 6. After an incubation period, single rinse pipettor 16 can inject a rinse buffer to stop the capture reagent binding reaction with precise timing. A substantial amount of the suspended fluo-bead can then be localized by magnets within the reaction rotor 6 over a period of time. After the magnets have substantially localized the fluo-beads within the cuvette, multi rinse pipettor 12 can aspirate and dispose of a portion of the rinse buffer, leaving a portion of the fluo-beads localized within the cuvette. Multi rinse pipettor 12 can proceed to inject a wash buffer into the cuvette of reaction rotor 6, resuspending the fluo-beads. The fluo-beads can again be localized by the magnets within reaction rotor 6 to be followed by multi rinse pipettor 12 aspirating and discarding a portion of the sample that was not localized from the cuvette in the reaction rotor 6. Thus, any unbound capture reagent is removed from the cuvette.

A patient sample can be contained in a sample tube in sample rotor 18. The patient sample can further be partially diluted with a sample diluent. At this point, sample pipettor 20 can aspirate a portion of the patient sample and inject the patient sample into the cuvette of reaction rotor 6 to resuspend the fluo-beads. The cuvette containing the patient sample within the reaction rotor 6 can then incubate the patient sample. In one embodiment, for example, the incubation temperature can be about 37° C.+/−about 0.2° C., while the incubation time can be about 37.75 minutes+/−about 2 minutes. After incubation, multi rinse pipettor 12 can inject the rinse buffer to again resuspend the fluo-beads. Another localization process is performed by reaction rotor 6 by allowing the fluo-beads to substantially collect within the cuvette near the magnets in reaction rotor 6. After the localization of the fluo-beads, multi rinse pipettor 12 can aspirate and discard a portion of the fluid within the cuvette of reaction rotor 6 that was not localized during the localization process.

Multiple rinse cycles can then be performed on the sample within the cuvette of reaction rotor 6. The rinse cycles can be performed using multi rinse pipettor 12 to inject a wash buffer into the cuvette to resuspend the fluo-beads. Another localization step can allow the fluo-beads to collect within the cuvette by the magnets within reaction rotor 6. After about a 90 second fluo-beads collection period, multi rinse pipettor 12 can aspirate and discard a portion of the wash buffer, leaving a substantial portion of the fluo-beads within the cuvette of the reaction rotor 6. Another rinse cycle can then occur using multi rinse pipettor 12 to again inject wash buffer into the cuvette and allow the fluo-beads to resuspend. Another fluo-bead localization process can utilize the magnets within the reaction rotor 6 to localize the fluo-beads from the rest of the sample. Finally, the multi rinse pipettor 12 can aspirate a portion of the sample that was not localized by the localization process.

At this point, R1 pipettor 4 can aspirate a conjugate contained in a conjugate cuvette within reagent rotor 14. R1 pipettor 4 can then inject the previously aspirated conjugate into the cuvette of the reaction rotor 6. After incubating the cuvette under controlled time and temperature in reaction rotor 6, multi rinse pipettor 12 can inject a rinse buffer into the cuvette in reaction rotor 6. Another fluo-bead localization cycle can be performed by allowing magnets within reaction rotor 6 to substantially localize the fluo-beads within the cuvette. Multi-rinse pipettor 12 can aspirate and discard a portion of the sample within the cuvette that has not been localized during the localization cycle.

Multiple-rinse cycles can be performed on the sample within the cuvette of reaction rotor 6. Multi rinse pipettor 12 can inject a wash buffer to resuspend the fluo-beads within the cuvette. Another fluo-bead localization cycle can localize the fluo-beads by locating the cuvette within close proximity to the magnets in reaction rotor 6 over an adequate period of time. After the localization cycle, multi rinse pipettor 12 can aspirate and discard a portion of the sample that was not localized during the localization cycle. Another wash cycle can then occur by using multi rinse pipettor 12 to inject the wash buffer to resuspend the fluo-beads. Another localization cycle can utilize the magnets within reaction rotor 6 to localize the fluo-beads within the cuvette. After the localization process, multi rinse pipettor 12 can again aspirate and discard a portion of the sample that was not localized during the localization cycle.

At this point, R2 pipettor 22 can aspirate a portion of a first substrate and a second substrate from reagent rotor 14 and inject the substrates into the mixed substrate container 24 creating a mixed substrate sample. R2 pipettor 22 can then aspirate the mixed substrate sample from the mixed substrate container 24 and inject the mixed substrate sample into the cuvette of the reaction rotor 6, resuspending the fluo-bead with the mixed substrate sample. The sample is then incubated for a period of time. The sample in the cuvette of reaction rotor 6 can then be aspirated by optics pipettor 8 and placed in optics device 10. After optics device 10 makes fluorescence and luminescence optical observations, the sample is discarded and the multi rinse pipettor rinses the cuvettes of reaction rotor 6 in preparation for the next test.

For the above steps to be possible, one ore more capture reagent must be bound to the fluo-beads within a cuvette in reaction rotor 6 to create a single solid phase that is then combined with a patient sample. In an embodiment of the present disclosure, a user of automated immunochemistry analyzer 1 can customize a solid phase on the fly with several different capture reagents using a recommendation based on a disease profile database 52 accessed by a control unit of automated immunochemistry analyzer 1 or a control unit in communication with automated immunochemistry analyzer 1.

In an embodiment, automated immunochemistry analyzer 1 can include a graphical user interface (“GUI”) 30 and a control unit 32 that work together to allow a user to customize a solid phase. GUI 30 and control unit 32 can accompany or be a part of automated immunochemistry analyzer 1, or can be located remotely from automated immunochemistry analyzer 1 and communicate with automated immunochemistry analyzer 1 via a wireless or wired data connection. In another embodiment, GUI and control unit 32 can be entirely separate from automated immunochemistry analyzer 1.

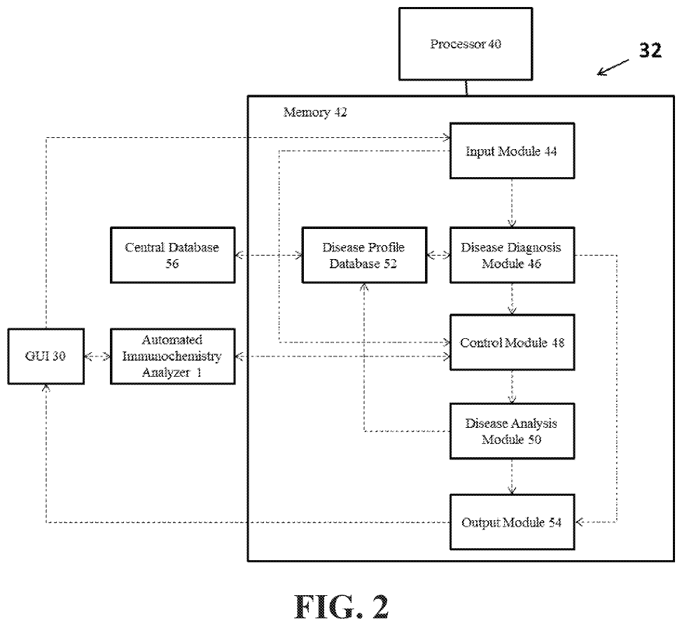

FIG. 2 illustrates an embodiment of control unit 32. As illustrated, control unit 32 can include a processor 40 and a memory 42, which can include a non-transitory computer readable medium. Memory 42 can include, for example, an input module 44, a disease diagnosis module 46, a control module 48, a disease analysis module 50, and an output module 54. Processor 40 can run the modules 44, 46, 48, 50, 52 in accordance with instructions stored on memory 42. The broken lines in FIG. 2 illustrate electrical connections between the modules 44, 46, 48, 50, 52 of control unit 32 and other elements such as automated immunochemistry analyzer 1, GUI 30 and/or central database 56. It should be understood by those of ordinary skill in the art that the illustrated modules and/or additional modules can be connected to the elements shown and/or additional internal or external elements.

As illustrated, memory 42 includes a disease profile database 52 that can be used by control unit 32 to recommend particular assays to run on a patient sample to test for particular diseases. In an embodiment, disease profile database 52 can be updated regularly by a wireless or wired connection to a central database 56. By updating disease profile database 52 regularly via central database 56, it can be ensured that recommendations made by control unit 32 using disease profile database 52 take the most up to date medical information into account. In an alternative embodiment of FIG. 2, disease profile database 52 can be included in a separate memory that is accessible to processor 40 and/or memory 42.

As explained in more detail below, disease profile database 52 enables control unit 32 to diagnose a patient's symptoms or provide prognostic information and recommend via GUI 30 that the patient be tested for certain diseases. As used herein, a “disease” can be, for example, an allergy, an infectious disease, metabolic disorder, injury or other medical condition. Moreover, “disease” as used herein can also include any health related index that is associated with an acute or chronic health condition or a predisposing or prognostic factor related to any of the preceding. The existence of a “disease” such as an allergy or an infection can be determined, for example, by the existence of allergens, infectious disease antigens and autoantigens. As used herein, a “symptom” can refer to any physical or mental feature that is regarded as indicating a condition of a disease, including, but not limited to, current symptoms, past symptoms, or the occurrence of another disease or medical condition that may be indicative of an additional disease.

Exemplary allergens include, but are not limited to, food allergens (i.e., peanuts, soy, shellfish, etc.), plant allergens (i.e., pollen, poison oak, grasses, weeds, trees, etc.), insect allergens (i.e., bee venom, etc.), animal allergens (i.e., wool, fur, dander, etc.), drugs (i.e. penicillin, sulfonamides, salicylates, etc.), mold spores, fragrances, latex, metals, wood, etc.

Exemplary infectious agents include, but are not limited to, bacteria, viruses, viroids, prions, nemotodes (e.g., roundworms, pinworms), parasites (e.g., malaria, tapeworm), and fungi (e.g., yeast, ringworm). As used herein the terms “infectious agent,” “pathogen”, “pathogenic microorganism” and “microorganism” all refer to the infectious agents listed above. Antigens from infectious agents can be any isolated protein, glycoprotein, nucleic acid, enzyme, lipid, liposaccharide, or combination thereof, from an infectious agent. Antigens can also include extracts or homogenized preparations from infectious agents which include a plurality of different antigenic moieties.

Exemplary autoantigens include, but are not limited to, nuclear antigens (target of antinuclear antibodies (ANA)), aggrecan, alanyl-tRNA syntetase (PL-12), alpha beta crystallin, alpha fodrin (Sptan 1), alpha-actinin, α1 antichymotrypsin, α1 antitripsin, α1 microglobulin, alsolase, aminoacyl-tRNA synthetase, amyloid (e.g., amyloid beta, amyloid P), annexins (e.g., annexin II, annexin V), apolipoproteins (e.g., ApoB, ApoE, ApoE4, ApoJ), aquaporin (e.g., AQP1, AQP2, AQP3, AQP4), bactericidal/permeability-increasing protein (BPI), β-globin precursor BP1, β-actin, β-lactoglobulin A, β2-glycoprotein I, β2-microglobulin, blood group antigens (e.g., Rh blood group antigens, I blood group antigens, ABO blood group antigens), C reactive protein (CRP), calmodulin, calreticulin, cardiolipin, catalase, cathepsin B, centromere proteins (e.g., CENP-A, CENP-B), chondroitin sulfate, chromatin, collagen (e.g., types I, II, III, IV, V, VI collagen), complement components (e.g., C1q, C3, C3a, C3b, C4, C5, C6, C7, C8, C9), cytochrome C, cytochrome P450 2D6, cytokeratins, decorin, dermatan sulfate, DNA (e.g., double stranded DNA, single stranded DNA), DNA topoisomerase I, elastin, Epstein-Barr nuclear antigen 1 (EBNA1), elastin, entaktin, extractable nuclear antigens (Ro, La, Sm, RNP, Scl-70, Jo1), Factor I, Factor P, Factor B, Factor D, Factor H, Factor X, fibrinogen (e.g., fibrinogen IV, fibrinogen S), fibronectin, formiminotransferase cyclodeaminase (LC-1), gliadin and amidated gliadin peptides (DGPs), gp210 nuclear envelope protein, GP2 (major zymogen granule membrane glycoprotein), glycoprotein glial fibrillary acidic protein (GFAP), glycated albumin, glyceraldehyde 3-phosphate dehydrogenase (GAPDH), haptoglobin A2, heat shock proteins (e.g., Hsp60, HSP70), hemocyanin, heparin, histones (e.g., histones H1, H2A, H2B, H3, H4), histidyl-tRNA synthetase (Jo-1), hyaluronidase, immunoglobulins, insulin, insulin receptor, integrins (e.g., integrins α₁β₁, α₂β₁, α₃β₁, α₄β₁, α₅β₁, α₆β₁, α₇β₁, α_{L}β₂, α_{M}β₂, α_{IIb}β₃, α_{V}β₁, α_{V}β₃, α_{V}β₅, α_{V}β₆, αVβ₈, α₆β₃, α₁β₁, interstitial retinol-binding protein 3, intrinsic factor, Ku (p70/p80), lactate dehydrogenase, laminin, liver cytosol antigen type 1 (LC1), liver/kidney microsomal antigen 1 (LKM1), lysozyme, melanoma differentiation-associated protein 5 (MDA5), Mi-2 (chromodomain helicase DNA binding protein 4), mitochondrial proteins (e.g., M1, M2, M3, M4, M5, M6, M7, M8, M9, BCOADC-E2, OGDC-E2, PDC-E2), muscarinic receptors, myelin-associated glycoprotein, myosin, myelin basic protein, myelin oligodendrocyte glycoprotein, myeloperoxidase (MPO), rheumatoid factor (IgM anti-IgG), neuron-specific enolase, nicotinic acetylcholine receptor α chain, nucleolin, nucleoporin (e.g., Nup62), nucleosome antigen, PM/Scl100, PM/Scl 75, pancreatic β-cell antigen, pepsinogen, peroxiredoxin 1, phosphoglucose isomerase, phospholipids, phosphotidyl inositol, platelet derived growth factors, polymerase beta (POLB), potassium channel KIR4.1, proliferating cell nuclear antigen (PCNA), proteinase-3, proteolipid protein, proteoglycan, prothrombin, recoverin, rhodopsin, ribonuclease, ribonucleoproteins (e.g., Ro, La, snRNP, scRNP), ribosomes, ribosomal phosphoproteins (e.g., P0, P1, P2), RNA (double stranded RNA, single stranded RNA), Sm proteins (e.g., SmB, SmB′, SmD1, SmD2, SmD3, SmF, SmG, SmN), Sp100 nuclear protein, SRP54 (signal recognition particle 54 kDa), selectin, smooth muscle proteins, sphinomyelin, streptococcal antigens, superoxide dismutase, synovial joint proteins, T1F1 gamma collagen, threonyl-tRNA synthetase (PL-7), tissue transglutaminase, thyroid peroxidase, thyroglobulin, thyroid stimulating hormone receptor, transferrin, triosephosphate isomerase, tubulin, tumor necrosis alpha, topoisomerase, U1-dnRNP 68/70 kDa, U1-snRNP A, U1-snRNP C, U-snRNP B/B′, ubiquitin, vascular endothelial growth factor, vimentin, and vitronectin.

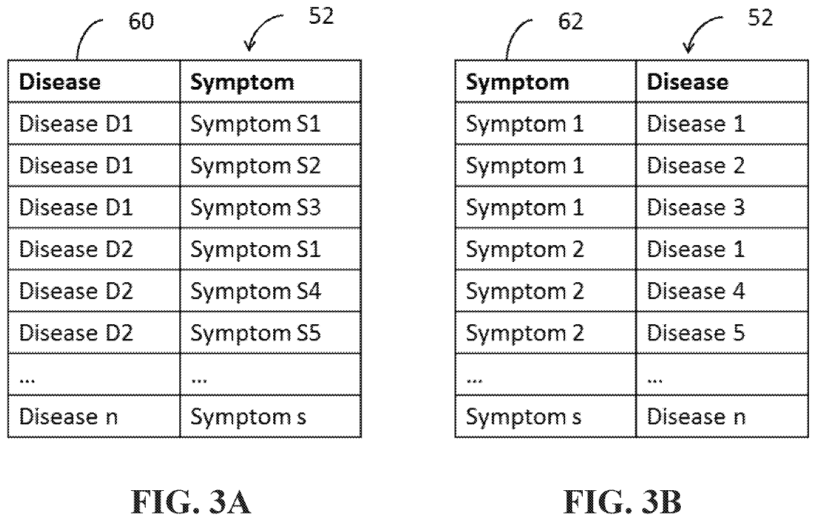

FIG. 3A shows one embodiment of a disease profile database 52 that allows a recommendation to be made based on an inputted symptom. In FIG. 3A, disease profile database 52 comprises a table 60 including a plurality of diseases and a plurality of corresponding symptoms. As illustrated, table 60 shows that disease D1 typically exhibits symptom 51, symptom S2 and symptom S3, while disease D2 typically exhibits symptom 51, symptom S4 and symptom S5. It should be understood that any number of diseases and symptoms can be listed in table 60, as indicated by disease n and symptom s in the last row of table 60.

In one example, a user inputs symptom 51 into GUI 30, and GUI 30 communicates the input to input module 44 of control unit 32. Disease analysis module 46 of control unit 32 then accesses table 60 and determines that symptom 51 is a symptom of both disease D1 and disease D2. Output module 54 of control unit 32 then causes GUI 30 to display both disease D1 and disease D2 as potential diseases that could cause symptom 51, and/or to recommend particular assays be performed for disease D1 and/or D2 using capture reagents stored by analyzer 1.

In another example, a user inputs symptom S2 into GUI 30, and GUI 30 communicates the input to input module 44 of control unit 32. Disease analysis module 46 of control unit 32 then accesses table 60 and determines that symptom 51 is a symptom of disease D1 but not disease D2. Output module 54 of control unit 32 then causes GUI 30 to display disease D1 as potential disease that could cause symptom S1, and/or to recommend a particular assay be performed for disease D1 using a capture reagent stored by analyzer 1.

In another example, a user inputs symptom S1 and symptom S2 into GUI 30, and GUI 30 communicates the input to input module 44 of control unit 32. Disease analysis module 46 of control unit 32 then accesses table 60 and determines that symptom S1 is a symptom of both disease D1 and disease D2, and that symptom S2 is a symptom of disease D1 but not disease D2. In one embodiment, output module 54 of control unit 32 then causes GUI 30 to display both disease D1 and disease D2 as potential diseases that could cause symptom S1, and/or to recommend particular assays be performed for disease D1 and/or D2 using capture reagents stored by analyzer 1. In another embodiment, disease analysis module 46 of control unit 32 eliminates disease D2 as a possibility due to symptom S2, and output module 54 of control unit 32 causes GUI 30 to display disease D1 as potential disease that could cause symptoms S1 and S2, and/or to recommend a particular assay be performed for disease D1 using a capture reagent stored by analyzer 1. In yet another embodiment, output module 54 of control unit 32 causes GUI 30 to display both disease D1 and disease D2 as potential diseases, but also alerts the user that disease D1 is more likely than disease D2 due to the occurrence of symptom S2, and/or recommends that particular assays be performed for disease D1 and/or D2 using capture reagents stored by analyzer 1.

FIG. 3B shows an alternative embodiment of a disease profile database 52 comprising a table 62 which shows potential diseases sorted according to potential symptoms.

In one example, a user inputs symptom S1 into GUI 30, disease analysis module 46 of control unit 32 accesses table 62 to determine that symptom S1 causes disease D1, disease D2 and disease D3, and output module 54 of control unit 32 causes GUI 30 to display disease D1, disease D2 and disease D3 as potential diseases that could cause symptom S1 and/or to recommend particular assays be performed for disease D1, disease D2 and/or disease D3 using capture reagents stored by analyzer 1.

In another example, a user inputs symptom S1 and symptom S2 into GUI 30, and disease analysis module 46 of control unit 32 accesses table 62 to determine that symptom S1 causes disease D1 and disease D2 and symptom S2 causes disease D1. Output module 54 of control unit 32 can then either cause GUI 30 to display both disease D1 and disease D2 as potential diseases that could cause symptom S1, cause GUI 30 to display disease D1 as potential disease that could cause symptoms S1 and S2, or cause GUI 30 to display both disease D1 and disease D2 as potential diseases and alert the user that disease D1 is more likely than disease D2 due to the occurrence of symptom S2. Output module 54 of control unit 32 can also cause GUI 30 to recommend particular assays be performed for disease D1 and/or disease D2 using capture reagents stored by analyzer 1.

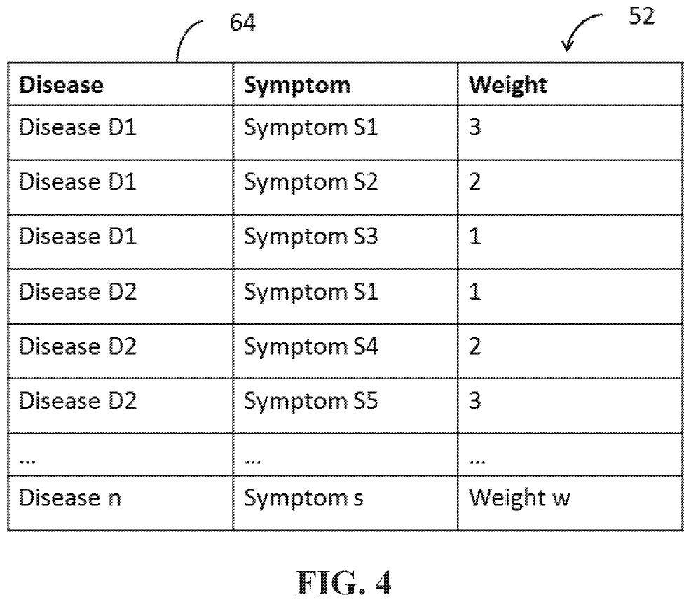

In an embodiment, the symptoms stored in disease profile database 32 can be weighted to show which symptoms are more likely to be indicative of a certain disease than other symptoms. FIG. 4 shows a disease profile database 52 comprising a table 64 with weighted symptoms. In FIG. 4, symptom S1 has a weight of 3, symptom S2 has a weight of 2, symptom S3 has a weight of 1, symptom S4 has a weight of 1, symptom S5 has a weight of 2, and symptom S6 has a weight of 3. It should be understood that any number of diseases, symptoms and weights can be listed in table 64, as indicated by disease n, symptom s and weight w in the last row of table 64.

In one example, a user inputs symptom S1 into GUI 30, and GUI 30 communicates the input to input module 44 of control unit 32. Disease analysis module 46 of control unit 32 then accesses table 64 and determines that symptom S1 is a symptom of both disease D1 and disease D2. Since symptom S1 has a weight of 3 for disease D1 and a weight of only 1 for disease D2, disease analysis module 46 of control unit 32 can determine that disease D1 is more likely to occur from symptom S1 than disease D2. Output module 54 of control unit 32 then causes GUI 30 to display both disease D1 and disease D2, but to indicate that disease D1 is more likely the cause of symptom S1 than disease D2. Output module 54 of control unit 32 can also cause GUI 30 to recommend particular assays be performed for disease D1 and/or D2 using capture reagents stored by analyzer 1.

In another example, a user inputs symptom S1 and symptom S4 into GUI 30, and GUI 30 communicates the input to input module 44 of control unit 32. Disease analysis module 46 of control unit 32 then accesses table 64 and determines that symptom S1 is a symptom of both disease D1 and disease D2, and that symptom S4 is a symptom of disease D2 but not disease D1. Disease analysis module 46 of control unit 32 then determines that both disease D1 and disease D2 are potential causes of the patient's symptoms because symptom S1 has a weight of 3 for disease D1, while symptoms S1 and S4 have a total weight of 3 (1+2) for disease D2. Output module 54 of control unit 32 then causes GUI 30 to display both disease D1 and disease D2 as potential diseases that could cause the patient's symptoms. Output module 54 of control unit 32 can also cause GUI 30 to recommend particular assays be performed for disease D1 and/or D2 using capture reagents stored by analyzer 1.

In another example, a user inputs symptom S1 and symptom S5 into GUI 30, and GUI 30 communicates the input to input module 44 of control unit 32. Disease analysis module 46 of control unit 32 then accesses table 64 and determines that symptom S1 is a symptom of both disease D1 and disease D2, and that symptom S5 is a symptom of disease D2 but not disease D1. Disease analysis module 46 of control unit 32 then determines that both disease D1 and disease D2 are potential causes of the patient's symptoms. Disease analysis module 46 further determines that symptom S1 has a weight of 3 for disease D1, while symptoms S1 and S4 have a total weight of 4 (1+3) for disease D2. Output module 54 of control unit 32 then causes GUI 30 to display both disease D1 and disease D2 as potential diseases that could cause the patient's symptoms, but to indicate that disease D2 is more likely the cause of the patient's symptoms than disease D2 due to the higher total weight applied to D2. Output module 54 of control unit 32 can also cause GUI 30 to recommend particular assays be performed for disease D1 and/or D2 using capture reagents stored by analyzer 1.

It should be understood that whole numbers have been used in the example of table 64 to simply the example, but it is envisioned that much more complicated weighting schemes could be used with the present disclosure. In another embodiment, fractional numbers and negative numbers could be used as weights. In yet another embodiment, weights can be applied to diseases/symptoms based on the individual patient's medical history or lifestyle and/or answers inputted into GUI 30 to particular questions regarding the patient's medical history and/or lifestyle.

In another example embodiment, weights could change depending on the symptoms. For example, if more than one symptom related to a particular disease is entered into GUI 30, disease analysis module 46 of control unit 32 could increase the weights related to that disease. In the above example in which the user inputs symptom S1 and symptom S4 into GUI 30, disease analysis module 46 of control unit 32 could determine that, because disease D2 shows both symptoms S1 and S4, the total weight for disease D2 or the individual weight for symptoms of disease D2 should be multiplied by 1.5. Disease analysis module 46 of control unit 32 could then determine that both disease D1 and disease D2 are potential causes of the patient's symptoms because symptom S1 has a weight of 3 for disease D1, while symptoms S1 and S4 have a total weight of 4.5 (1.5×(1+2)) for disease D2. Output module 54 of control unit 32 could then cause GUI 30 to display both disease D1 and disease D2 as potential diseases that could cause the patient's symptoms, but to indicate that disease D2 is more likely the cause of the patient's symptoms than disease D2 due to the higher total weight applied to D2. Output module 54 of control unit 32 could also cause GUI 30 to recommend particular assays be performed for disease D1 and/or D2 using capture reagents stored by analyzer 1.

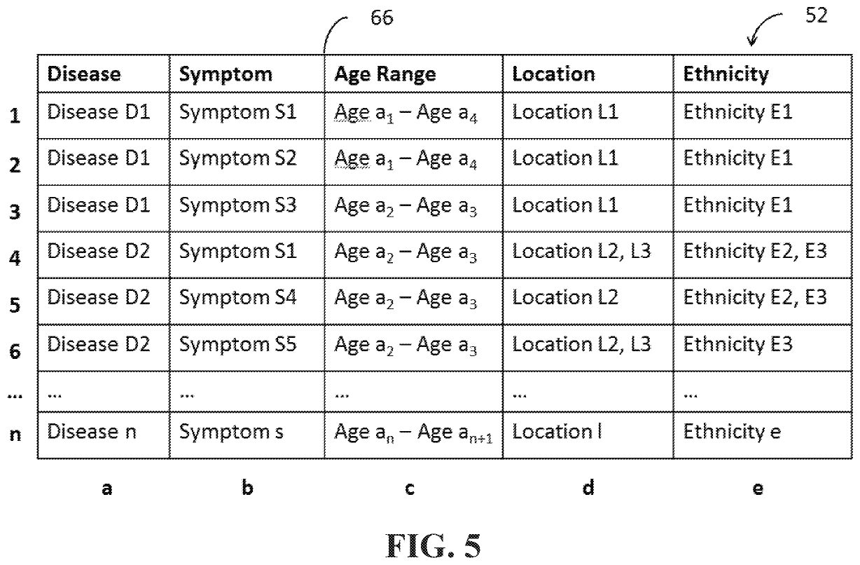

In addition to symptoms, other patient information entered into GUI 30 can be taken into account in determining potential diseases. FIG. 5 shows a disease profile database 52 comprising a table 66 that includes a patient age range, patient location and patient ethnicity. In the illustrated embodiments, age a₁ is the lowest age and age a₄ is the highest age, with age a₂ and age a₃ representing ages between age a₁ and age a₄. It should be understood that any number of diseases, symptoms, ages, locations and ethnicities can be listed in table 66, as indicated by disease n, symptom s, age aₙ-age aₙ₊₁, location l and ethnicity e in the last row of table 66.

Using table 66, disease analysis module 46 of control unit 32 can recommend diseases/assays to a user via GUI 32 based on a combination of elements as described above. It should be understood that not all elements of a disease need to be met for disease analysis module 46 of control unit 32 to recommend that a disease be tested for.

In one example, a user inputs symptom S1, an age range of age a₂ to age a₃, a location L1 and an ethnicity E2 into GUI 32, and GUI 30 communicates the input to input module 44 of control unit 32. Symptom S1 occurs with both disease D1 and disease D2, age range age a₂ to age a₃ corresponds with both disease D1 and disease D2, location L1 corresponds to disease D1, and ethnicity E2 corresponds to D2. It is not out of the question, however, that ethnicity E1 could contract disease D2 or that disease D1 could be contracted at location L1. Disease analysis module 46 via output module 50 therefore could cause GUI 30 to display both disease D1 and disease D2 as potential diseases that could cause the patient's symptom. Output module 54 of control unit 32 could also cause GUI 30 to recommend particular assays be performed for disease D1 and/or D2 using capture reagents stored by analyzer 1.

In an embodiment, disease analysis module 46 of control unit 32 can cause GUI 30 to prompt questions to the user in response to the analysis of table 66 by control unit 32. For example, in the above case, disease analysis module 46 via output module 54 can cause GUI 30 to ask the user whether the patient has recently visited location L2 or location L3. If the answer is yes, disease analysis module 46 can determine that the patient meets all of the criteria of disease D2 and output module 54 can cause GUI 30 to recommend that the patient be tested for disease D2. Output module 54 can also cause GUI 30 to recommend that the patient be tested for disease D1 and disease D2, and optionally indicate that disease D2 is believed to be more likely. Output module 54 can also cause GUI 30 to recommend particular assays be performed for disease D1 and/or D2 using capture reagents stored by analyzer 1.

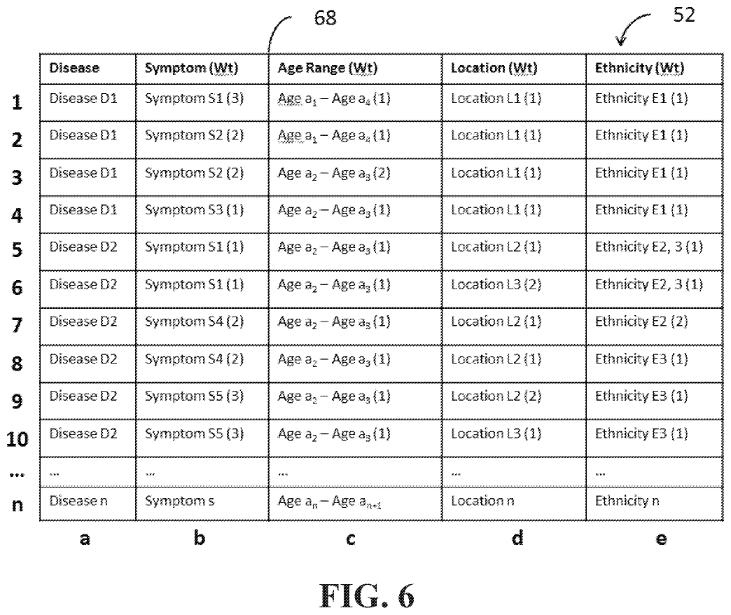

As set forth above, each of the inputs listed in table 66 can be weighted to allow control unit 32 to make a recommendation of diseases to test for. FIG. 6 shows a disease profile database 52 including a table 68 with weighted symptoms, age ranges, locations and ethnicities. In table 68, for example, a patient with disease D1 is more likely to show symptom S2 if the patient's age range is between age a₂ and age a₃ as opposed to between age a₁ and age a₄ (compare c3 with c2), a patient with disease D2 is more likely to show symptom S4 if the patient's ethnicity is ethnicity E2 as opposed to ethnicity E3 (compare e7 with e8), and a patient with disease D2 is more likely to show symptom S1 if the patient is located in location L3 as opposed to location L2 (compare d6 with d5).

In one example embodiment, a user inputs symptom S1 and symptom S4, an age range between age a₂ and age a₃, a location L1 and an ethnicity E2, and GUI 30 communicates the input to input module 44 of control unit 32. In an embodiment, disease analysis module 46 of control unit 32 could give disease D1 a total weight of 5 (3 from symptom S1 (b1), 1 from the age range between age a₂ and age a₃ (which are a subgroup of age a₁ to age a₄) (c1), 1 from location L1 (d1)), and could give disease D2 a total weight of 8 (1 from symptom S1 (b5, b6), 1 from symptom S1 being within the age range (c5, c6), 1 from symptom S1 corresponding to ethnicity E2 (e5), 2 for symptom S4 (b7, b8), 1 from symptom S4 being within the age range (c7, c8), and 2 from symptom S1 corresponding to ethnicity E2 (e7)). Output module 54 of control unit 32 can then cause GUI 30 to recommend that the patient be tested for disease D1 and disease D2, and optionally indicate that disease D2 is believed to be more likely based on its higher weight. Output module 54 of control unit 32 can also cause GUI 30 to recommend particular assays be performed for disease D1 and/or D2 using capture reagents stored by analyzer 1.

In another example embodiment with the same inputs, disease analysis module 46 of control unit 32 could calculate the total weight by multiplying individual weights across rows. For example, disease analysis module 46 of control unit 32 could give disease D1 a total weight of 3 (by multiplying 3×1×1) (3 from symptom S1 (b1), 1 from the age range between age a₂ and age a₃ (c1), 1 from location L1 (d1)), and could give disease D2 a total weight of 5 (1×1×1+2×1×2) (multiplying 2 from symptom S1 (b5), 1 for symptom S1 being within the age range (c5), and 1 for symptom S1 corresponding to ethnicity E2 (e5), and multiplying 2 for symptom S4 (b7), 1 for symptom S4 being within the age range (c7, c8), and 2 for symptom S1 corresponding to ethnicity E2 (e7)). Output module 54 of control unit 32 can then cause GUI 30 to recommend that the patient be tested for disease D1 and disease D2, and optionally indicate that disease D2 is believed to be more likely based on its higher weight. Output module 54 of control unit 32 can also cause GUI 30 to recommend particular assays be performed for disease D1 and/or D2 using capture reagents stored by analyzer 1.

It is envisioned that there are numerous ways to use weights to provide recommendations of which disease is more likely to be present in the patient. The weights can be applied to only one of the columns shown in tables 60, 62, 64, 66, 68 (e.g. only the symptom column), or can be applied to multiple columns. Those of ordinary skill in the art will recognize other ways to apply weights that can be used in accordance with the present disclosure.

As set forth above, output module 54 of control unit 32 can cause GUI 30 to recommend particular assays be performed for particular diseases using capture reagents stored by analyzer 1. In an embodiment, control unit 32 stores information regarding the plurality of capture reagents stored by analyzer 1, and can determine which capture reagents should used or mixed to test for one or more diseases. For example, control unit can determine that Capture Reagent A stored by analyzer 1 tests for disease D1 and that Capture Reagent B stored by analyzer 1 tests for disease D2. After advising via GUI 32 that the patient be tested for disease D1 and disease D2, control unit 32 can cause automated immunochemistry analyzer 1 to perform an assay by mixing Capture Reagent A with Capture Reagent B to test for both disease D1 and disease D2 simultaneously. GUI 32 can also give the user the option of performing one or both of disease D1 and/or disease D2, and control unit control unit 32 can cause automated immunochemistry analyzer 1 to perform the assay based on the user's instructions.

In an example embodiment to exemplify the above description, the symptom inputted into GUI could be a patient developing hives after drinking wine. Control unit 32 could analyze a disease profile database 52 and determine that the patient's symptom could be indicative of any one of a yeast allergy, a tartaric acid allergy, or a grape allergy. Each of these three allergies could then be displayed to the user via GUI 30, and the user may be enabled to select one or more of the three allergies for further testing. If all three of the allergies are selected for further testing, control unit 32 can instruct automated immunochemistry analyzer 1 to perform an assay by mixing the appropriate capture reagent to test for a yeast allergy, the appropriate capture reagent to test for a tartaric acid allergy, and the appropriate capture reagent to test for the grape allergy. Particular assays and/or particular capture reagents stored by analyzer 1 could also be displayed to the user via GUI 30, and the user may be enabled to select one or more of the assays/capture reagents.

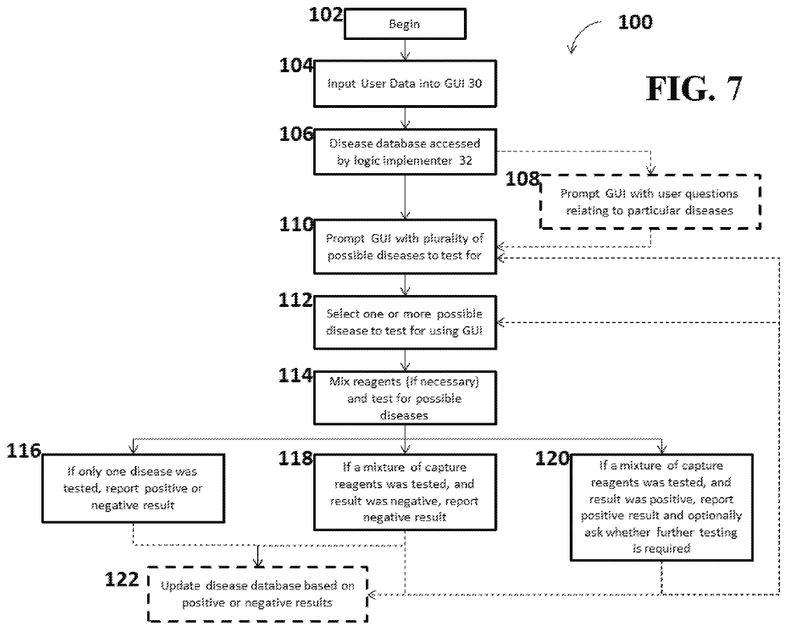

FIG. 7 illustrates a flow chart showing a process 100 using the disease profile database 52 as described above. Beginning at step 102, a user initiates the process by instructing GUI 30 that a patient wishes to be tested for one or more diseases. At step 104, the user inputs the patient's disease symptoms into GUI 30 along with any other patient characteristics such as age, location and ethnicity. Those of ordinary skill in the art will recognize other inputs that could be used to determine diseases that have not yet been mentioned herein, for example, height, weight, medications currently being taken, and other medical history characteristics. The inputs are then directly or indirectly communicated to input module 44 of control unit 32 by GUI 30.

At step 106, disease analysis module 46 of control unit 32 receives the inputs from GUI 30 via input module 44, accesses disease profile database 52, and determines one or more possible diseases that the patient should be tested for. Disease analysis module 46 can determine the possible diseases, for example, by accessing a table 60, 62, 64, 66, 68 using the user inputs and/or by weighting the user inputs to determine a disease as described above.

Optionally, at step 108, based on the analysis of disease profile database 52 by disease analysis module 46, disease analysis module 46 via output module 54 can cause GUI 30 to prompt questions about the patient to better determine the likelihood of certain diseases. For example, GUI can ask the user whether the patient has recently visited certain locations where particular diseases are more prevalent. GUI can also ask the user whether the patient has in the past shown certain symptoms or contracted other diseases which may indicate a higher likelihood of the patient currently suffering from one disease over another. GUI can also ask the user how often the patient comes into contact with an allergen source (e.g., “Does the patient own a cat?”). Disease analysis module 46 can then use the answers to the questions to evaluate the patient for potential diseases, for example, by applying weights to the diseases/symptoms based on the answers.

In the above example in which the patient developed hives after drinking wine, and in which control unit 32 determined that the patient's symptom could be indicative of any one of a yeast allergy, a tartaric acid allergy, or a grape allergy, control unit 32 could for example prompt questions on GUI 30 to narrow the possible allergy at issue. For example, control unit 32 could prompt GUI 30 to ask whether the patient has also exhibited any allergic reaction when consuming bread, which would indicate that the yeast allergy is most likely to be the patient's issue.

At step 110, based on the analysis at step 106, and optionally the analysis of the responses to questions at step 108, disease analysis module 46 via output module 54 can cause GUI 30 to display one or more possible diseases caused by the patient's symptoms and/or one or more assays that should be run on the patient sample using particular capture reagents stored by analyzer 1. At step 112, the user can select one or more of the prompted possible diseases/assays for testing by inputting commands into GUI. In an embodiment, the user can also request that additional unprompted diseases be tested.

At step 114, the user selection is communicated to control module 48 of control unit 32 via input module 44. Control module 48 then communicates with automated immunochemistry analyzer 1 to cause automated immunochemistry analyzer 1 to perform, in situ, the requested assays (test for the requested diseases). In an embodiment, control module 48 of control unit 32 communicates with a separate control unit of automated immunochemistry analyzer 1, and the separate control unit of automated immunochemistry analyzer 1 causes the individual elements of automated immunochemistry analyzer 1 to function to perform the requested assays and test for the requested diseases.

If multiple diseases are to be tested, control module 48 can cause automated immunochemistry analyzer 1 to mix, in situ, a plurality of capture reagents from reaction rotor 14 to create a single solid phase that tests a patient sample for reactivity to multiple immunogens. In an embodiment, control module 48 determines where each of the selected capture reagents is located within reagent rotor 14 of automated immunochemistry analyzer 1, causes automated immunochemistry analyzer 1 to dispense the appropriate amount of fluo-beads into a cuvette located within the reaction rotor 6, and then controls R1 pipettor 4 and reagent rotor 14 to cause R1 pipettor 4 to aspirate each of the individually selected capture reagents from reagent rotor 14 and inject the capture reagents into the cuvette located in reaction rotor 6. In another embodiment, control module 48 causes the capture reagents to be mixed prior to being injected into a cuvette.

The combination solid phase is then combined with a patient sample and incubated, bound and tested as described above by performing several wash steps, adding the conjugate and substrate, and then aspirating the patient sample into optics pipettor 8 so that optics device 10 can take fluorescence and luminescence measurements. The fluorescence and luminescence measurements are then communicated to disease analysis module 50 of control unit 32, which analyzes the results as being positive or negative for one or more particular disease. Disease analysis module 50 then communicates the results via output module 54 to GUI 30.

As understood by those of ordinary skill in the art, a positive result determined for a mixture of capture reagents indicates a positive result for at least one of the capture reagents in the mixture. For example, a positive test with respect to a mixture containing Capture Reagent A, Capture Reagent B, and Capture Reagent C would indicate a positive test for at least one of Capture Reagent A, Capture Reagent B, and Capture Reagent C. In this case, however, it may not be possible to determine which one or more of Capture Reagent A, Capture Reagent B, and Capture Reagent C caused the positive test. On the other hand, a negative result for a mixture of Capture Reagent A, Capture Reagent B, and Capture Reagent C conclusively indicates that the patient sample did not test positive for any one of Capture Reagent A, Capture Reagent B, and Capture Reagent C.

If only a single capture reagent was used to test for a single disease, then at step 116, output module 54 of control unit 32 can cause GUI 30 to display the positive or negative result of the test as determined by disease analysis module 50. Alternatively, the positive or negative result can be reported to the user via another reporting mechanism.

If a mixture of capture reagents was used to test for multiple diseases, and if disease analysis module 50 recorded a negative result for the mixture of capture reagents, then at step 118, output module 54 of control unit 32 can cause GUI 30 to display the negative result of the test determined by disease analysis module 50, meaning that the patient tested negative for each of the tested diseases. Alternatively, the negative result can be reported to the user via another reporting mechanism.

If a mixture of capture reagents was used to test for multiple diseases, and if disease analysis module 50 recorded a positive result for the mixture of capture reagents, then at step 120, output module 54 of control unit 32 can cause GUI 30 to display the positive result of the test determined by disease analysis module 50, meaning that the patient tested positive for at least one of the tested diseases. In an optional embodiment, GUI 30 can then ask whether further testing is required to determine which of the diseases caused the positive response. If the user desires further testing, then the process can return to step 112 and control module 48 of control unit 32 can either break down the capture reagents into subgroups, or test each individual capture reagent separately, depending on how many capture reagents are in the combination or how many capture reagents could have yielded the positive result. Once the additional testing of the capture reagents has been performed, and disease analysis module 50 has determined whether each capture reagent results in a positive or negative test, output module 54 of control unit 32 can report to the user via GUI 30 or another reporting mechanism the results for each capture reagent of the combination.

In an embodiment, a positive test or negative for a particular disease can cause control unit 32 to recommend that other particular diseases be tested for. For example, a positive result at step 116 or step 120 or a negative result at step 118 can cause control unit 32 to reevaluate other potential diseases and make an additional recommendation to the user at step 110. The method can then proceed by performing the additional testing at steps 112 and 114 based on the additional recommendation.

In an optional embodiment, disease analysis module 50 of control unit 32 can access disease profile database 52 and update disease profile database 52 at step 122 based on the positive or negative results determined from the testing at step 114. For example, if a patient tests positive for a certain disease, disease profile database 52 can be updated to reflect the positive test for the disease corresponding to the patient's age, location, ethnicity, symptoms or other factors. By performing step 122 by continuously updating disease profile database 52, control unit 32 can provide the most up-to-date recommendations to future patients, for example, by tracking when diseases are spread amongst new ages, locations, ethnicities and other factors or by tracking new symptoms. In an embodiment, disease analysis module 50 of control unit 32 can access disease profile database 52 and update disease profile database 52 at step 122 by changing the weights applied to different factors. For example, if a series of tests come back positive for a certain age, location, ethnicity or symptom, then the weight applied to that age, location, ethnicity or symptom can be increased to reflect the increasing likelihood of the occurrence of the disease based on that factor.

By running a mixture of capture reagents, the amount of capture reagent stored by automated immunochemistry analyzer 1 can be significantly reduced. It should be understood that the tests are being run for hundreds or thousands of samples, and that every negative test of a multiple reagent mixture requires only one test (versus multiple individual tests) to determine that the patient sample tests negatively for each of the multiple capture reagents. The reduction of the total number of tests is significant as the number of capture reagents tested is increased.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the disclosure and does not pose a limitation on the scope of the disclosure otherwise claimed. No language in the specification should be construed as indicating any non-claimed element essential to the practice of the disclosure.

The use of the term “or” in the claims is used to mean “and/or” unless explicitly indicated to refer to alternatives only or the alternatives are mutually exclusive, although the disclosure supports a definition that refers to only alternatives and “and/or.”

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of the disclosure are described herein, including the best mode known to the inventors for carrying out the disclosure. Of course, variations on those preferred embodiments will become apparent to those of ordinary skill in the art upon reading the foregoing description. The inventor expects those of ordinary skill in the art to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

Further, it is to be understood that the embodiments of the disclosure disclosed herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

The invention is claimed as follows:

1. A system for diagnosing and testing a patient for one or more disease, the system comprising:
an assay apparatus storing a plurality of capture reagents, the assay apparatus configured to perform a plurality of different assays on a patient sample using the plurality of capture reagents;
a user interface in operable communication with the assay apparatus, the user interface configured to allow input of at least one patient symptom; and
a control unit in operable communication with the user interface, the control unit configured to (i) analyze a disease profile database based on the at least one patient symptom inputted into the user interface, (ii) output at least one recommended assay using at least one of the plurality of capture reagents stored by the assay apparatus based on the analysis of the disease profile database, and (iii) cause the assay apparatus to perform the at least one assay using the at least one of the plurality of capture reagents.

2. The system of claim 1, wherein the control unit is configured to cause the assay apparatus to perform the at least one assay by: (i) isolating the at least one of the plurality of capture reagents based on the at least one recommended assay; (ii) binding the at least one of the plurality of capture reagents to paramagnetic particles, (iii) binding analyte molecules from the patient sample to the at least one of the plurality of capture reagents, and (iv) analyzing the bound analyte molecules from the patient sample to determine a positive or negative result for the at least one recommended assay.

3. The system of claim 1, wherein the at least one disease profile database is configured to output a plurality of recommended assays to be performed by the assay apparatus using two or more of the plurality of capture reagents stored by the assay apparatus.

4. The system of claim 3, wherein the controller is configured to cause the assay apparatus to perform each of the plurality of recommended assays separately.

5. The system of claim 3, wherein the controller is configured to cause the assay apparatus to perform each of the plurality of recommended assays simultaneously.

6. The system of claim 5, wherein the controller is configured to cause the automated immunochemistry analyzer to: (i) mix the two or more of the plurality of capture reagents together; (ii) bind the mixture of the two or more of the plurality of capture reagents to the paramagnetic particles, (iii) bind analyte molecules from the patient sample to the bound mixture of the two or more of the plurality of capture reagents, and (iv) analyze the bound analyte molecules from the patient sample.

7. The system of claim 1, wherein each capture reagent is specific for an immunogen selected from the group consisting of allergens, infectious disease antigens and autoantigens.

8. The system of claim 1, wherein the control unit stores locations of the plurality of capture reagents within the assay apparatus, and controls the assay apparatus to perform the at least one assay by retrieving the at least one of the plurality of capture reagents from a corresponding stored location.

9. A method for diagnosing and testing a patient for one or more disease, the method comprising:
inputting a patient's symptoms into a user interface;
analyzing at least one patient symptom by accessing a disease profile database;
displaying on the user interface at least one disease that may be present in the patient based on the analysis of the at least one patient symptom;
requesting at least one test be performed on a patient sample to test for the at least one disease;
receiving a result of the at least one test; and
treating the patient for the at least one disease if the result of the at least one test indicates that the patient sample tested positive for the at least one disease.

10. The method of claim 9, wherein inputting the patient's symptoms into the user interface includes answering questions about the patient prompted on the user interface.

11. The method of claim 9, wherein displaying on the user interface at least one disease includes displaying on the user interface a plurality of diseases, and wherein performing the at least one assay includes mixing a plurality of capture reagents and performing a plurality of assays simultaneously to determine the occurrence of the plurality of diseases.

12. A system for diagnosing and testing a patient for one or more disease, the system comprising:
a user interface configured to allow input of at least one patient symptom;
a disease profile database stored on a non-transitory computer readable medium, the disease profile database linking a plurality of diseases to a plurality of patient symptoms;
a control unit configured to (i) analyze the disease profile database using the inputted at least one patient symptom, (ii) cause the user interface to display at least one recommended disease to test for the patient based on the analysis, and (iii) allow selection of the at least one recommended disease via the user interface for further testing.

13. The system of claim 12, wherein the control unit is configured to determine at least one capture reagent to be mixed with a patient sample to perform at least one assay to test for the at least one disease.

14. The system of claim 12, wherein the control unit is configured to cause the user interface to display a plurality of recommended diseases to test for the patient based on the analysis.

15. The system of claim 14, wherein the control unit is configured to cause an assay apparatus to test for the plurality of recommended diseases simultaneously by mixing a plurality of capture reagents together.

16. The system of claim 14, wherein the user interface is configured to allow selection of one or more tests for one or more of the plurality of recommended diseases.

17. The system of claim 14, wherein the user interface is configured to indicate that one of the plurality of recommended diseases is more likely to be present than another of the plurality of recommended diseases based on the inputted at least one patient symptom.

18. The system of claim 17, wherein the control unit determines the likelihood of the plurality of recommended diseases based on weights assigned to patient symptoms within the disease profile database.

19. The system of claim 12, which includes an automated immunochemistry analyzer, and wherein the control unit is configured to control the automated immunochemistry analyzer to perform the further testing for the at least one disease using at least one capture reagent stored by the immunochemistry analyzer.

20. The system of claim 19, wherein the control unit stores locations of a plurality of capture reagents within the automated immunochemistry analyzer, and controls the automated immunochemistry analyzer to test for the at least one disease by retrieving the at least one capture reagent from a corresponding stored location.

21. A method for diagnosing and testing a patient for one or more disease, the method comprising:
inputting a patient's symptoms into a user interface;
analyzing at least one patient symptom by accessing a disease profile database;
displaying on the user interface at least one disease that may be present in the patient based on the analysis of the at least one patient symptom;
determining at least one assay to be performed on a patient sample to test for the at least one disease;
selecting at least one capture reagent from a plurality of selectable capture reagents to test for the at least one disease; and
performing the at least one assay using the at least one capture reagent.

22. The method of claim 21, wherein performing the at least one suggested assay includes:
adding the at least one capture reagent to a container containing paramagnetic particles;
binding the at least one capture reagent to the paramagnetic particles;
binding analyte molecules from the patient sample to the at least one capture reagent; and
analyzing the bound analyte molecules from the patient sample to determine a positive or negative result for the at least one assay.
